(19) United States
(12) Patent Application Publication
(10) Pub. No.: US 2026/0341433 A1
(43) Pub. Date: Oct. 29, 2026

(54) Artificial Intelligence [AI] Assisted Anesthesia Monitoring and Decision Support Systems

(71) Applicant: Fouad Yassa, Clermont, FL (US)

(72) Inventor: Fouad Yassa, Clermont, FL (US)

(21) Appl. No.: 19/649,322

(22) Filed: Apr. 16, 2026

Related U.S. Application Data
(60) Provisional application No. 63/793,583, filed on Apr. 23, 2025.

(51) Int. Cl. G16H 10/60; A61B 5/00; G16H 20/10

(52) U.S. Cl. CPC G16H 10/60; A61B 5/0024; G16H 20/10

(57) ABSTRACT

An anesthesia decision support system and method are disclosed. The system receives patient specific clinical data and real-time physiologic monitoring data. The system aggregates the patient specific clinical data and the physiologic monitoring data and generates a patient specific anesthetic plan. During an anesthetic procedure, the system continuously monitors and performs real-time analysis of the physiologic monitoring data, detects an event based on the physiologic monitoring data, generates a ranked list of candidate causes for the event, and generates a recommended intervention corresponding to at least one candidate cause. The system presents the anesthetic plan, the ranked list, and the recommended intervention on a display. In some embodiments, the system receives clinician input selecting confirm, modify, or dismiss for a recommended intervention and records a case record entry indicating a clinician disposition for documentation. In some embodiments, the system uses a hybrid decision support framework including programmed algorithms with clinical thresholds, a knowledge base containing anesthesia protocols and dosing guidance, and machine learning models trained using historical anesthesia case data. In some embodiments, the system receives a clinician query through an input interface and presents a generated response based on at least the patient specific clinical data and the physiologic monitoring data.

### CROSS-REFERENCE TO RELATED APPLICATIONS

This application claims the benefit of U.S. Provisional Patent Application No. 63/793,583, filed Apr. 23, 2025, the entire contents of which are incorporated herein by reference.

### BACKGROUND OF THE INVENTION

### Field of the Invention

This disclosure relates generally to perioperative patient monitoring and anesthesia care support systems. More particularly, this disclosure relates to computer implemented clinical monitoring and decision-support systems configured to receive and process physiologic monitoring data and patient specific clinical data and to assist clinicians in evaluating patient status during anesthesia care.

### Background

Anesthesia care involves planning, monitoring, and documentation across a procedure. The anesthesia provider reviews patient history and test results. The anesthesia provider selects drugs and monitoring. The anesthesia provider then monitors the patient during the procedure. The anesthesia provider also responds to changes in patient condition.

Many data sources exist during perioperative care. Patient history often sits in an EHR [electronic health record] or EMR [electronic medical record]. Intraoperative signals arrive from multiple monitors. Examples include ECG [electrocardiogram], blood pressure, SpO2 [peripheral capillary oxygen saturation], and EtCO2 [end tidal carbon dioxide]. Ventilator settings and infusion data may appear on separate screens. The anesthesia provider often correlates these sources mentally. This can require frequent screen switching and manual correlation during time-sensitive moments.

Current monitoring devices display numbers, waveforms, and alarms. Alarms often use fixed thresholds. The alarms may lack context about the patient and case phase. Frequent alarms can create fatigue. The provider may spend time sorting signal from noise. As a result, clinicians may need to interpret isolated alarms without integrated context from related parameters or patient factors.

Planning tools also vary in quality and scope. Many tools rely on templates and checklists. The tools may not adapt to changing physiology. The tools may not account for interactions across multiple parameters. Some tools focus on a single risk or single phase. Other tools focus on retrospective review after the case ends. As a result, pre-procedure planning and intra-procedure monitoring may remain disconnected during evolving intraoperative conditions.

When patient status changes, the provider forms a differential diagnosis. The provider also selects an intervention. This work can occur under time pressure. Dose selection can require quick calculations. The provider may also need to confirm contraindications and allergies. The provider may then document events and actions. Documentation can be delayed during urgent moments.

These limitations can increase cognitive load during anesthesia care. These limitations can also lead to variability between providers. There remains a need for improved perioperative support systems that reduce screen switching and manual correlation, provide more context for interpretation of physiologic changes, and support more consistent documentation during time-sensitive events.

### SUMMARY OF THE INVENTION

This disclosure describes an anesthesia decision support system. The system supports perioperative anesthesia care. The system supports planning before a procedure. The system supports monitoring during a procedure. The system supports recommendations during a procedure. The system supports reporting after a procedure. In some embodiments, the system supports generation of a ranked list of candidate causes for detected physiologic changes and generation of recommended interventions for clinician review. In some embodiments, the system supports clinician initiated queries and presentation of generated responses based on patient context.

In some embodiments, the system includes a processor and a memory storing instructions. The system includes a display. The system includes a data interface configured to receive patient specific clinical data and physiologic monitoring data. The system may be implemented as a built-in monitor integrated with an anesthesia machine. The system may be implemented as an external monitor positioned near an anesthesia machine. In some embodiments, the external monitor includes a tablet or workstation display.

In some embodiments, the system aggregates patient specific clinical data and physiologic monitoring data. The system generates a patient specific anesthetic plan based on at least the patient specific clinical data. The system continuously monitors and performs real-time analysis of the physiologic monitoring data during an anesthetic procedure. The system detects an event based on the real-time analysis of the physiologic monitoring data. The system generates a ranked list of candidate causes for the event. The system generates a recommended intervention corresponding to at least one candidate cause. The system presents the anesthetic plan, the ranked list, and the recommended intervention on the display. In some embodiments, the system presents supporting information associated with the ranked list, including one or more physiologic parameters or detected trends that contributed to ranking.

In some embodiments, the system uses a hybrid decision support framework. The framework may include programmed algorithms with clinical thresholds. The framework may include a knowledge base containing anesthesia protocols and dosing guidance. The framework may include machine learning models trained using historical anesthesia case data. In some embodiments, the system applies safety checks using patient specific clinical data including at least allergy data or contraindication data before presenting or finalizing a recommended intervention.

In some embodiments, the system receives clinician input selecting confirm, modify, or dismiss for a recommended intervention. The system records a case record entry indicating a clinician disposition for the recommended intervention. In some embodiments, the system generates a post procedure report summarizing planning outputs, detected events, presented recommendations, and clinician dispositions. In some embodiments, the system presents planning outputs, monitoring outputs, and recommendations in an integrated interface to reduce screen switching and manual correlation. In some embodiments, the post procedure report is configured for export to an EHR [electronic health record] system, an EMR [electronic medical record] system, or an AIMS [anesthesia information management system].

### BRIEF DESCRIPTION OF DRAWINGS

FIG. 1 illustrates an example architecture of an anesthesia decision support system (100) configured to receive patient data inputs (110), procedure data inputs (112), and real-time physiologic monitoring data via a data acquisition unit (114), and to process the inputs using modules including an anesthetic plan module (120), a monitoring and analysis module (122), a differential diagnosis module (124), a recommendation engine (126), and an emergence and post-procedure module (128), with outputs presented through a clinician user interface (130) and optionally accessed through an interactive query interface (132).

FIG. 2 illustrates a data collection layer (200) configured to receive and aggregate data through a manual input interface (210), an integration platform interface (212), a direct device interface (214), and an EMR system interface (216), and to process the data using a data normalization module (220) for downstream analysis by the anesthesia decision support system (100).

FIG. 3 illustrates an AI anesthetic plan generation arrangement in which patient data (110) and surgical procedure data (112) are processed by an AI anesthetic plan generator (120) to produce a generated anesthetic plan (300) including anesthesia type selection (310), preoperative medication recommendations (320), pre-procedure evaluation recommendations (330), advanced monitoring and tools checklist (340), fluid management recommendations (350), induction plan recommendations (360), and intraoperative plan recommendations (370).

FIG. 4 illustrates a real-time monitoring interface (400) including data collection sources (410), vital signs and ventilator panel (420), trend graphs and visualization panel (430), alerts and anomaly highlights panel (440), suggested diagnosis panel (450), recommended interventions panel (460), system navigation menu (470), and clinical information display (480).

FIG. 5 illustrates a differential diagnosis panel (500) triggered by anomaly detection (501) and configured to present ranked diagnoses including a first diagnosis block (510), a second diagnosis block (520), and a third diagnosis block (530), each including corresponding probability indicators (511, 521, 531), supporting evidence fields (512, 522, 532), patient factors fields (513, 523, 533), expected findings fields (514, 524, 534), and recommended interventions fields (515, 525, 535).

FIG. 6 illustrates a real-time recommendation interface (600) responsive to a differential diagnosis (500), the interface (600) including a recommended drug or intervention module (610), a dosing calculation module (620), a justification for recommendation module (630), and a clinician action and confirmation module (640).

FIG. 7 illustrates an emergence plan interface (700) including preparation before emergence (710), a transition phase (720), an extubation readiness checklist (730), an extubation strategy (740), and immediate post-extubation care (750).

FIG. 8 illustrates a PACU [post anesthesia care unit]/ICU [intensive care unit] handoff report interface (800) including patient and procedure identification (810), anesthetic and intraoperative course (820), current status on arrival to PACU [post anesthesia care unit]/ICU [intensive care unit] (830), and postoperative considerations (840).

FIG. 9 illustrates an interactive clinician query workflow including selected patient context (900), user initiation of an “Ask the AI” query (910), a query input interface (920), an AI query processing engine (930), a generated response or recommendation interface (940), and clinician review and action (950).

### DETAILED DESCRIPTION OF THE INVENTION

### Overview

This disclosure describes an artificial intelligence [AI] assisted anesthesia monitoring and decision support system. The system supports anesthesia care across a pre-procedure phase, an intra-procedure phase, and a post-procedure phase. The system receives patient specific clinical data and real-time physiologic monitoring data. The system processes the data to generate planning outputs, monitoring insights, differential diagnoses, and recommended interventions for clinician review. In many embodiments, the system presents recommendations as advisory suggestions and supports clinician confirmation, modification, or dismissal before documentation.

The system can be implemented as a built-in component of an anesthesia machine display or as an external modular device positioned near an anesthesia machine. The external device can be a tablet, a workstation, or another display device. The system can be used in an operating room or another procedure suite.

### System Architecture and Data Flow

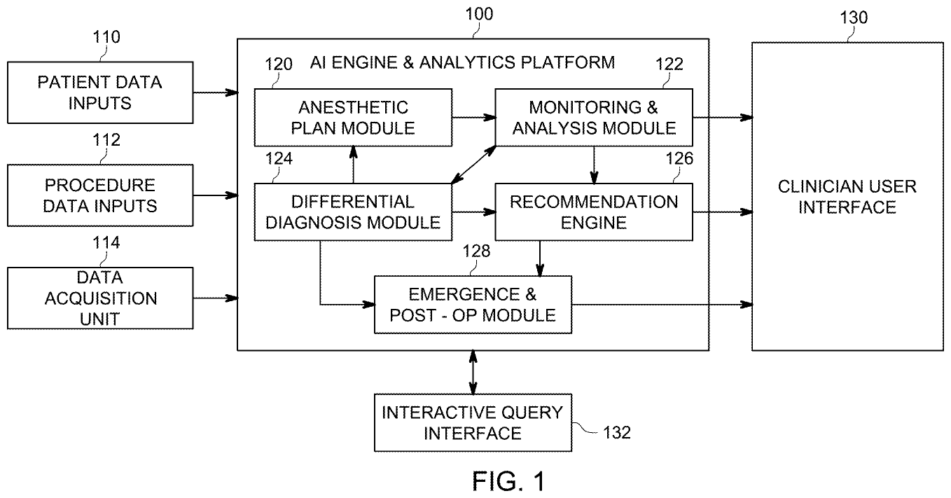

Referring to FIG. 1, an AI engine and analytics platform (100) receives data from multiple sources and produces outputs for a clinician. Patient data inputs (110) can include demographics, medical history, medication history, allergy data, laboratory values, and similar pre-procedure information. Procedure data inputs (112) can include procedure type, expected duration, surgical context, and other case level information.

A data acquisition unit (114) receives real-time physiologic monitoring data. The physiologic monitoring data can include values, waveforms, or both. Examples include ECG [electrocardiogram] data, blood pressure data, SpO2 [peripheral capillary oxygen saturation] data, EtCO2 [end tidal carbon dioxide] data, and ventilator related data. The data acquisition unit (114) can receive data through direct device connections, through a hospital integration platform, or through combinations of sources described below. An anesthetic plan module (120) generates an anesthesia plan using one or more of the patient data inputs (110) and the procedure data inputs (112). A monitoring and analysis module (122) analyzes real-time physiologic monitoring data received through the data acquisition unit (114). When an event, deviation, or anomaly is detected, a differential diagnosis module (124) generates candidate causes and ranks the candidate causes. A recommendation engine (126) generates recommended interventions corresponding to one or more candidate causes. [0027] An emergence and post-procedure module (128) supports workflow during emergence and post-procedure transition. A clinician user interface (130) presents planning outputs, monitoring outputs, ranked diagnoses, and recommended interventions. An interactive query interface (132) supports clinician initiated questions, including voice or text inputs, to retrieve context aware information and recommendations.

### Data Collection and Normalization

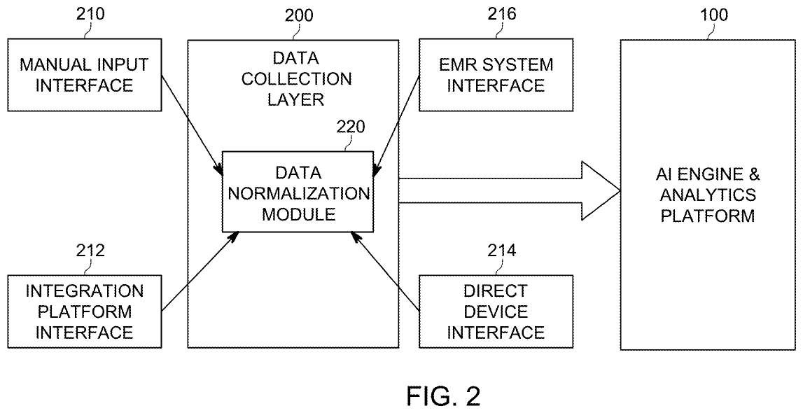

Referring to FIG. 2, a data collection layer (200) can aggregate data from heterogeneous sources and route data into the AI engine and analytics platform (100). A manual input interface (210) can accept clinician entered information that is not automatically captured. Examples include qualitative observations, procedural events, or workflow notes.

An integration platform interface (212) can communicate with a device integration platform or middleware that aggregates device streams. A direct device interface (214) can communicate with medical instrumentation using device drivers, device APIs, serial interfaces, or network interfaces. An EMR system interface (216) can communicate with an EHR [electronic health record] or EMR [electronic medical record] system using standard data exchange approaches such as HL7 [Health Level Seven] and FHIR [Fast Healthcare Interoperability Resources], where available.

A data normalization module (220) can align data streams in time, normalize units, reduce artifacts, and map input data into a common internal representation. This allows information from different sources to be evaluated together. In some embodiments, the data normalization module (220) flags missing values, indicates data freshness, or tracks source identifiers for later audit and documentation.

### AI Based Anesthetic Plan Generation

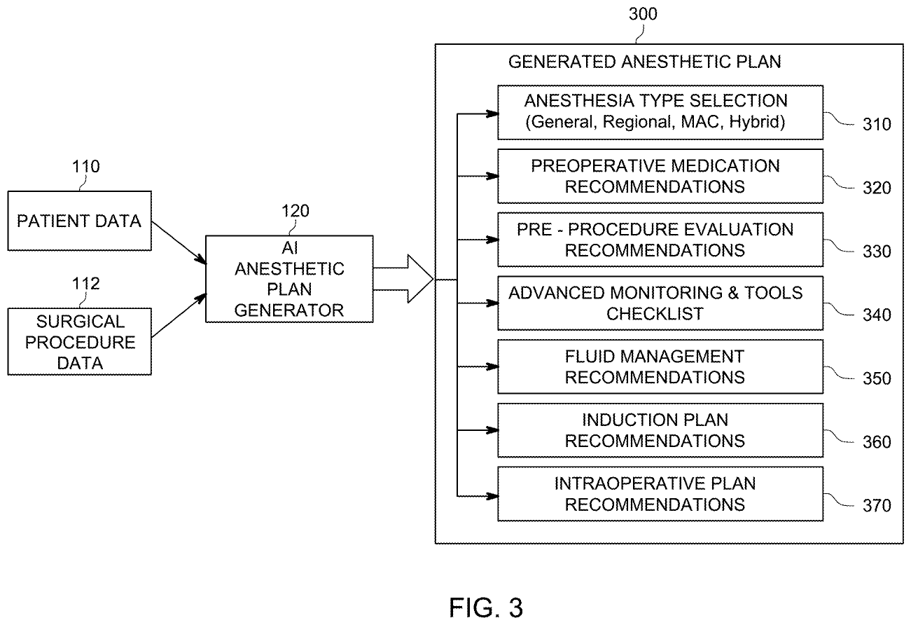

Referring to FIG. 3, the AI anesthetic plan generator (120) receives the patient data inputs (110) and the procedure data inputs (112) and generates a plan output. A generated anesthetic plan (300) can include structured plan sections that a clinician can review and modify.

An anesthesia type selection (310) can identify a recommended anesthesia technique, such as general anesthesia, regional anesthesia, monitored anesthesia care, or combinations. A preoperative medication recommendations section (320) can identify medications or medication classes for pre-procedure use. A pre-procedure evaluation recommendations section (330) can identify evaluations or workups relevant to the planned procedure and patient status. An advanced monitoring and tools checklist (340) can identify monitoring approaches and equipment needs. A fluid management recommendations section (350) can provide planning guidance for anticipated fluid management. An induction plan recommendations section (360) can provide a structured approach for induction agents and related steps. An intraoperative plan recommendations section (370) can provide planning guidance for maintenance strategy, ventilation strategy, and intra-procedure goals.

The generated anesthetic plan (300) can be updated as additional information becomes available. For example, updated laboratory values, changes in procedure information, or intra-procedure events can be used to revise parts of the plan. In many embodiments, the clinician user interface (130) supports review and modification of plan components before or during a case.

### Real-Time Monitoring Interface and Workflow

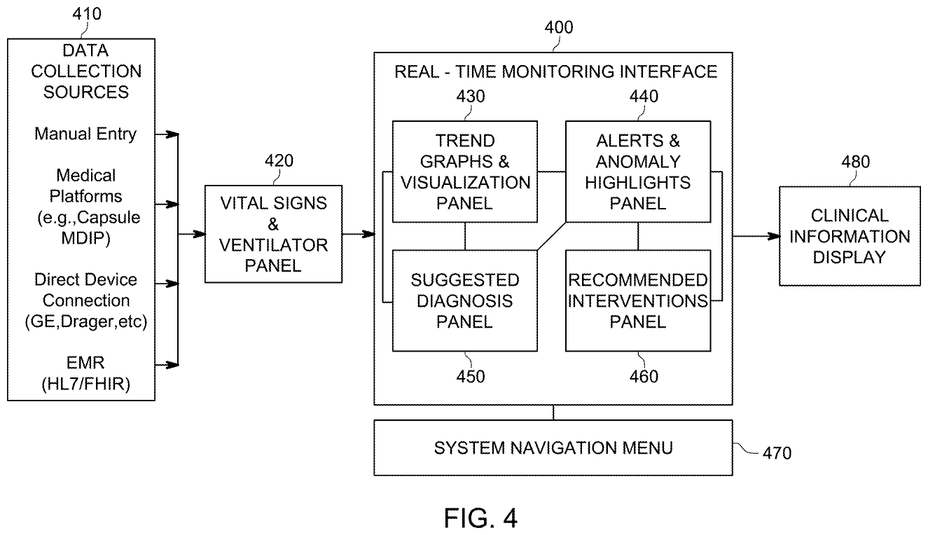

Referring to FIG. 4, a real-time monitoring interface (400) presents an integrated dashboard for intra-procedure use. Data collection sources (410) represent the pathways by which the system receives data, including manual entry, integration platforms, direct device connections, and EHR or EMR interfaces.

A vital signs and ventilator panel (420) can present physiologic parameters and ventilator related parameters. A trend graphs and visualization panel (430) can present historical trajectories, trends, and responses to prior interventions. An alerts and anomaly highlights panel (440) can present deviations from baselines, trends, or thresholds with context. A suggested diagnosis panel (450) can present ranked candidate causes derived from the differential diagnosis module (124). A recommended interventions panel (460) can present suggested actions generated by the recommendation engine (126). A system navigation menu (470) can support switching between views such as planning, monitoring, differential diagnosis, recommendations, emergence, reporting, and query workflows. A clinical information display (480) represents the output hardware on which the interface is rendered.

In some embodiments, the monitoring and analysis module (122) evaluates physiologic monitoring data using multiple approaches. The evaluation can include threshold detection, trend detection over time windows, deviation from a patient specific baseline, and multi-parameter pattern recognition. In some embodiments, an anomaly detection approach includes statistical analysis and can also include a machine learning model trained on historical anesthesia case data. The monitoring and analysis module (122) can generate a confidence value associated with detected events and use the confidence value to prioritize display in the alerts and anomaly highlights panel (440).

### Differential Diagnosis Interface

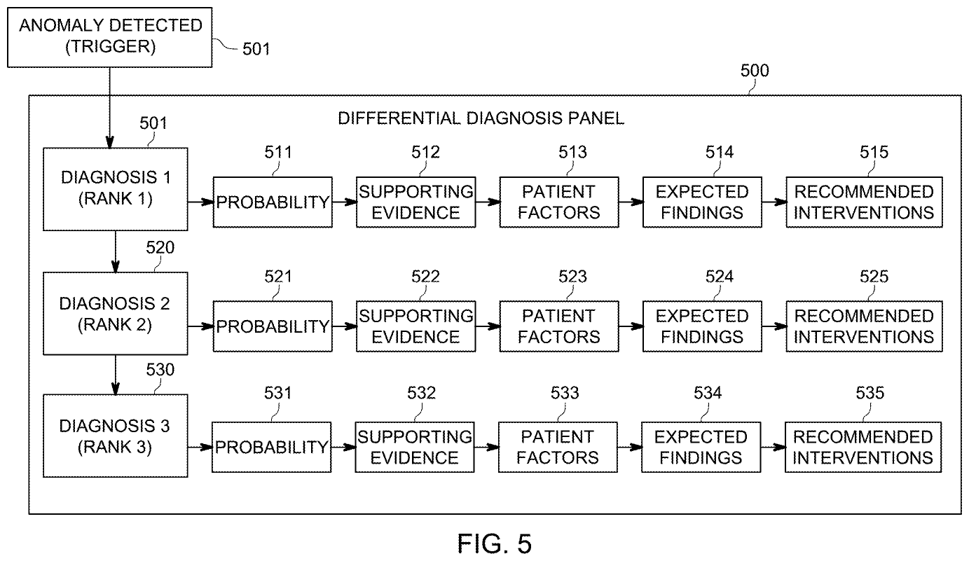

Referring to FIG. 5, a differential diagnosis interface (500) can be rendered when an anomaly is detected (501) or when a deviation from expected parameters is detected. The interface can present a ranked hierarchy of candidate causes. The interface can include a first diagnosis block (510), a second diagnosis block (520), and a third diagnosis block (530). These blocks can be ordered based on calculated probability scores, confidence values, or both.

The first diagnosis block (510) can include a probability indicator (511) and a physiologic evidence field (512) describing the signals or trends that contributed to the diagnosis ranking. The first diagnosis block (510) can include patient specific factors (513) that relate a candidate cause to patient history, comorbidities, medications, or other context. The first diagnosis block (510) can include expected additional findings (514) that prompt clinician checks or confirmation steps. The first diagnosis block (510) can include recommended interventions (515) that can be selected to move into the recommendation workflow.

The second diagnosis block (520) can include a probability indicator (521), a physiologic evidence field (522), patient specific factors (523), expected additional findings (524), and recommended interventions (525). The third diagnosis block (530) can include a probability indicator (531), a physiologic evidence field (532), patient specific factors (533), expected additional findings (534), and recommended interventions (535).

In some embodiments, the differential diagnosis module (124) ranks candidate causes using rule-based logic, probabilistic modeling, pattern matching, machine learning inference, or combinations. The ranking can be influenced by procedure phase, recent interventions, and physiologic relationships across parameters.

### Real-Time Recommendation Interface and Clinician Confirmation

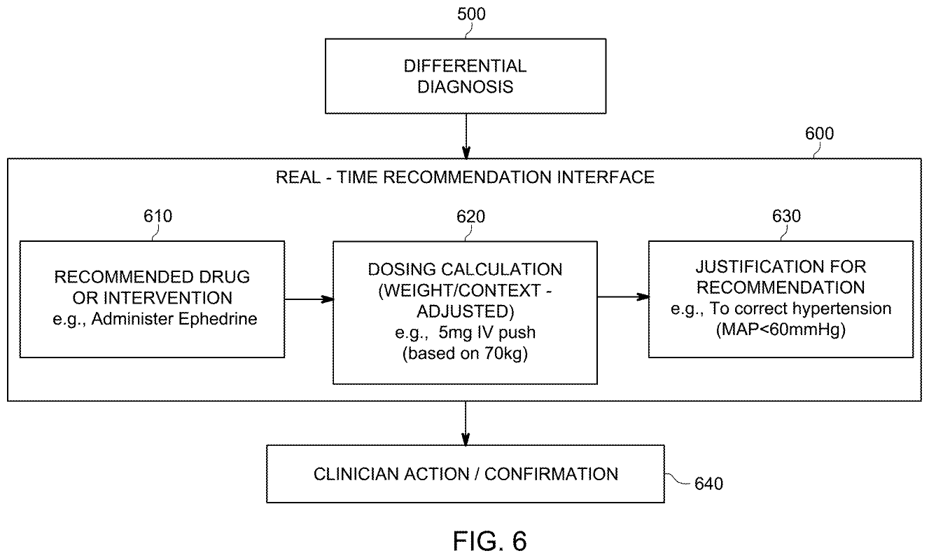

Referring to FIG. 6, a real-time recommendation interface (600) translates ranked diagnoses into actionable recommendations. A recommended drug or intervention module (610) can present a specific intervention, including pharmacologic actions or procedural actions. A dosing calculation module (620) can present a dose range or a patient specific calculated dose value. A justification for recommendation panel (630) can present an explainable rationale tied to monitored parameters, detected trends, or protocol logic. A clinician action and confirmation mechanism (640) can accept clinician input to confirm, modify, or dismiss a recommendation.

Dose recommendations can originate from more than one source. In some embodiments, dose recommendations draw from a protocol and dosing library, institution configured dosing standards, patient specific calculations, risk adjusted physiologic models, and machine learning inference derived from historical cases. The system can combine sources and present a dosing range, a calculated value, or both. In some embodiments, safety checks consider allergy data, contraindications, drug interactions, and institution configured limits before presenting or finalizing a recommendation. Clinician interaction can be recorded for documentation. In many embodiments, a confirmed or modified recommendation can be time stamped and stored as part of a case record entry. In some embodiments, dismissed recommendations can also be stored along with an optional clinician note.

### Emergence Plan Interface

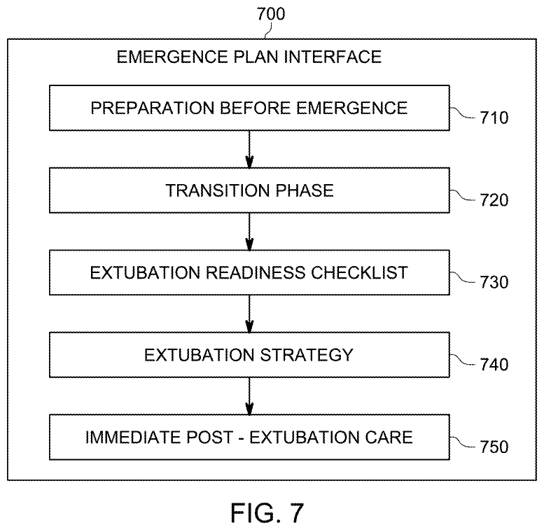

Referring to FIG. 7, an emergence plan interface (700) supports the transition from maintenance to emergence. A preparation before emergence module (710) can provide planning guidance for timing and readiness steps. A transition phase module (720) can provide guidance for transitions in ventilation support and monitoring emphasis. An extubation readiness checklist (730) can provide structured criteria for readiness evaluation. An extubation strategy module (740) can present decision support for airway removal strategy based on patient and procedural context. An immediate post-extubation care module (750) can provide guidance for monitoring and early detection of complications after airway removal. In some embodiments, the emergence plan interface (700) uses intra-procedure events, administered medications, and patient risk factors to tailor displayed checklists and guidance. In many embodiments, the emergence workflow remains advisory and supports clinician review and modification.

### PACU or ICU Handoff Reporting

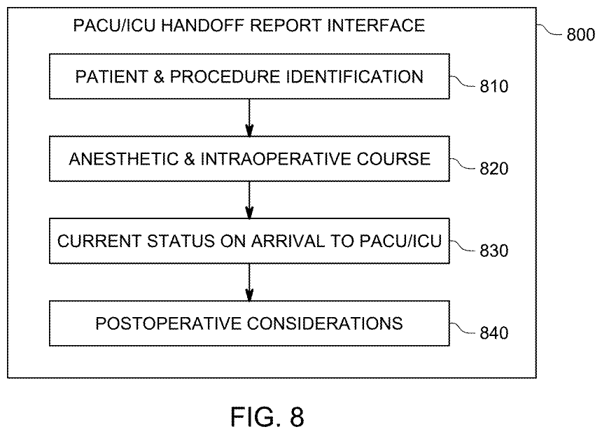

Referring to FIG. 8, a PACU [post anesthesia care unit] or ICU [intensive care unit] handoff report interface (800) can present a structured summary suitable for transfer of care. A patient and procedure identification section (810) can present patient identifiers and procedure identifiers. An anesthetic and intraoperative course section (820) can summarize key events and actions during the case. A current status on arrival section (830) can present the patient state at transfer. A postoperative considerations section (840) can present follow-on considerations and guidance.

In some embodiments, the report is generated based on case record entries created during the procedure, including confirmed, modified, or dismissed recommendations. In some embodiments, the report supports export to an EHR, an anesthesia information management system, or another clinical system through available interfaces.

### Interactive Query Workflow

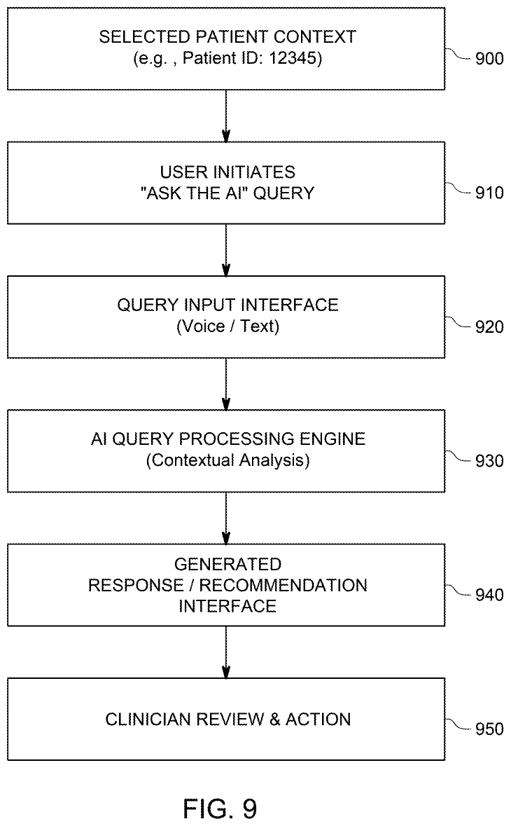

Referring to FIG. 9, an interactive query workflow supports clinician initiated questions. A selected patient context module (900) anchors responses to a selected patient record. A user initiates query step (910) can begin a voice or text query sequence. A query input interface (920) receives voice inputs, text inputs, or both. An AI query processing engine (930) performs contextual analysis using patient specific clinical data, procedure context, and current physiologic monitoring data. A generated response or recommendation interface (940) presents an output for clinician review. A clinician review and action step (950) allows acceptance, modification, or dismissal and can record interaction outcomes for documentation.

In some embodiments, the interactive query interface (132) can retrieve protocol guidance, dosing ranges, or monitoring context. In many embodiments, the query workflow presents outputs as advisory and supports clinician oversight.

### Hybrid Decision Support Framework

In some embodiments, the system uses a hybrid decision support framework that includes rule-based logic, a knowledge base containing anesthesia protocols and dosing guidance, and machine learning models trained on historical anesthesia case data. Rule-based logic can support thresholding, protocol mapping, and safety checks. The knowledge base can store protocols, dosing references, and institution configured standards. Machine learning models can support pattern recognition, anomaly detection, risk scoring, and ranking of candidate causes.

In some embodiments, the system applies weighting between rule-based logic and machine learning outputs. The weighting can depend on case context, available inputs, or institution preferences. In some embodiments, the system records model version identifiers and configuration settings for later review.

### Computer Readable Medium Implementation

In some embodiments, instructions for performing operations described in this disclosure are stored on a nontransitory computer readable storage medium. The storage medium can include semiconductor memory, magnetic storage, or optical storage. Examples include solid state storage, hard drives, and optical discs such as CD ROM and DVD.

### Deployment Examples

In some embodiments, the AI engine and analytics platform (100) runs locally within a facility network. In some embodiments, parts of the platform run on a local computing device positioned near an anesthesia machine. In some embodiments, parts of the platform run on a server accessible through a network connection. In some embodiments, a hybrid deployment uses an on-site component for real-time acquisition and display and uses a separate computing resource for additional processing. Data can be acquired through direct device integration, through a device integration platform, through an AIMS [anesthesia information management system], through an EHR or EMR system, or through combinations of these pathways.

1. An anesthesia decision support system comprising:
a processor;
a display;
a data interface configured to receive patient specific clinical data and physiologic monitoring data; and
a memory storing instructions that, when executed by the processor, cause the anesthesia decision support system to:
aggregate the patient specific clinical data and the physiologic monitoring data;
generate a patient specific anesthetic plan based on at least the patient specific clinical data;
continuously monitor and perform real-time analysis of the physiologic monitoring data during an anesthetic procedure;
detect an event based on the real-time analysis of the physiologic monitoring data;
generate a ranked list of candidate causes for the event;
generate a recommended intervention corresponding to at least one candidate cause based on the real-time analysis;
present, on the display, at least the patient specific anesthetic plan, the ranked list, and the recommended intervention;
receive clinician input selecting at least one of confirm, modify, or dismiss for the recommended intervention; and
record, in response to the clinician input, a case record entry indicating a clinician disposition for the recommended intervention.

2. A computer implemented method for anesthesia decision support comprising:
receiving patient specific clinical data and physiologic monitoring data;
aggregating the patient specific clinical data and the physiologic monitoring data;
generating a patient specific anesthetic plan based on at least the patient specific clinical data;
continuously monitoring and performing real-time analysis of the physiologic monitoring data during an anesthetic procedure;
detecting an event based on the real-time analysis of the physiologic monitoring data;
generating a ranked list of candidate causes for the event;
generating a recommended intervention corresponding to at least one candidate cause based on the real-time analysis;
displaying at least the patient specific anesthetic plan, the ranked list, and the recommended intervention;
receiving clinician input selecting at least one of confirm, modify, or dismiss for the recommended intervention; and
recording, based on the clinician input, a case record entry indicating a clinician disposition for the recommended intervention.

3. A nontransitory computer readable storage medium storing instructions that, when executed by a processor of an anesthesia decision support system, cause performance of the method of claim 2.

4. The anesthesia decision support system of claim 1 wherein the display is integrated with an anesthesia machine as a built-in display component, or is provided by an external monitor positioned adjacent the anesthesia machine.

5. The anesthesia decision support system of claim 1 wherein the data interface is configured to receive physiologic monitoring data from at least two physiologic monitors.

6. The anesthesia decision support system of claim 1 further comprising an input interface configured to receive at least one of manual clinician input, touch input, or voice input.

7. The anesthesia decision support system of claim 1 wherein detecting the event comprises detecting an absolute threshold condition in the physiologic monitoring data.

8. The anesthesia decision support system of claim 1 wherein detecting the event comprises detecting a trend over a time window in the physiologic monitoring data.

9. The anesthesia decision support system of claim 1 wherein detecting the event comprises detecting a deviation from a patient specific baseline.

10. The anesthesia decision support system of claim 1 wherein detecting the event comprises detecting a multi parameter pattern based on at least two physiologic parameters.

11. The anesthesia decision support system of claim 1 wherein generating the ranked list comprises assigning a confidence value to at least one candidate cause.

12. The anesthesia decision support system of claim 1 wherein the recommended intervention comprises a medication recommendation that includes a suggested dosing range.

13. The anesthesia decision support system of claim 12 wherein the medication recommendation further includes a patient specific calculated dose value.

14. The anesthesia decision support system of claim 12 wherein generating the medication recommendation is based on a protocol library stored in the memory.

15. The anesthesia decision support system of claim 12 wherein generating the medication recommendation is further based on institution configured dosing standards.

16. The anesthesia decision support system of claim 1 wherein generating the recommended intervention comprises evaluating at least one of an allergy indicator, a contraindication indicator, or a drug interaction indicator derived from the patient specific clinical data.

17. The anesthesia decision support system of claim 1 wherein the case record entry indicates whether the recommended intervention was confirmed, modified, or dismissed.

18. The anesthesia decision support system of claim 1 further comprising generating a post procedure report that includes at least a summary of the patient specific anesthetic plan, detected events, presented recommendations, and clinician dispositions.

19. The anesthesia decision support system of claim 1 wherein generating the ranked list or the recommended intervention uses a hybrid framework comprising rule-based logic and a machine learning model trained using historical anesthesia case data.

20. The anesthesia decision support system of claim 6 wherein the physiologic monitoring data includes waveform data obtained from at least one physiologic monitor, and wherein the anesthesia decision support system is configured to:
receive a clinician query through the input interface; and
present, on the display, a generated response based on at least the patient specific clinical data and the physiologic monitoring data.
